(19) United States
(12) Patent Application Publication
(10) Pub. No.: US 2004/0198686 A1
(43) Pub. Date: Oct. 7, 2004

(54) Regulation of allergen induced gene

(76) Inventor: Marc Elliot Rothenberg, Cincinnati, OH (US)

Correspondence Address: WOOD, HERRON & EVANS, LLP, 2700 CAREW TOWER, 441 VINE STREET, CINCINNATI, OH, 45202, US

(21) Appl. No.: 10/759,658

(22) Filed: Jan. 16, 2004

Related U.S. Application Data
(60) Provisional application No. 60/440,922, filed on Jan. 18, 2003.

(51) Int. Cl. A61K 48/00; A61K 38/19

(52) U.S. Cl. 514/44; 424/85.2

(57) ABSTRACT

A composition and method for alleviation of an allergic response by regulation of resistin-like molecules α or β (RELMα or RELMβ) expression. RELMS are also disclosed as markers for assessment of an allergic patient's status, for example, monitoring inflammation and/or tissue repair in a lung or airway of an asthmatic patient. Regulation of RELMS is involved in the pathogenesis of allergic lung inflammation and other allergen-induced conditions. The discovery of the involvement of RELMα and RELMβ in the asthma mechanistically links the pathogenesis of insulin resistance and obesity, known to involve RELMS, with asthma.

### RELATED APPLICATION

This application claims priority to United States Provisional Patent Application Serial No. 60/440,922 filed Jan. 18, 2003, now pending and expressly incorporated by reference herein in its entirety.

### REGULATION OF ALLERGEN INDUCED GENE

The U.S. Government has a paid-up license in this invention and the right in limited circumstances to require the patent owner to license others on reasonable terms as provided for by the terms of Grant No. RO1 A142242-04 awarded by the National Institutes of Health.

### FIELD OF THE INVENTION

The invention relates to compositions and methods to regulate expression of resistin-like molecules (RELM) α and β.

### BACKGROUND

Asthma is a complex chronic inflammatory pulmonary disorder. Despite intense research, the incidence of asthma is on the rise and it is the chief diagnosis responsible for pediatric hospital admissions.

Asthma research has largely focused on analysis of the cellular and molecular pathways induced by allergen exposure in sensitized animals, including humans. Studies have identified elevated production of IgE, mucus hypersecretion, airway obstruction, inflammation and enhanced bronchial reactivity to spasmogens in the asthmatic response. Clinical and experimental investigations have demonstrated a strong correlation between the presence of CD4⁺T helper 2 lymphocytes (Th2 cells) and disease severity, which suggested a role for these cells in the pathophysiology of asthma. Th2 cells are thought to induce asthma through the secretion of a variety of cytokines (IL-4, -5, -6, -9 -10, -13, -25) which activate inflammatory and residential effector pathways both directly and indirectly. IL-4 and IL-13 are produced at elevated levels in the asthmatic lung and are thought to be key regulators of many of its hallmark features.

Attention has recently focused on the pathogenesis of airway remodeling in the setting of chronic airway inflammation. Mesenchymal cell signaling, induced by Th2 cytokines, has an active role in chronic injury and repair processes in response to allergen triggered inflammation. Thus, multiple therapeutic agents likely interfere with specific inflammatory pathways, and the development of the asthma phenotype is likely to be related to the complex interplay of a large number of additional genes, and their polymorphic variants.

The resistin family of cytokines contains several conserved proteins having molecular weights in the range of 12.5 kDa with 10 or 11 cysteine residues that promote oligomeric forms. One family member is resistin, which is also called adipocyte-secreted factor (ADSF), or found in inflammatory zone 3 (F1ZZ3). Resistin is a novel hormone secreted by adipocytes, and may link obesity with insulin resistance and type 11 diabetes. Other family members are resistin-like molecules (RELM). RELMα was originally found in inflammatory zones in a murine model of experimental asthma, and subsequently designated F1ZZ1. RELMα is expressed in adipose tissue, heart, lung, and tongue, and RELMβ is expressed in the intestine. Recently, RELMy has been identified and shown to be expressed at highest levels in hematopoietic tissues.

Initial studies in mice suggested that resistin mediated insulin resistance by antagonizing insulin action and modulating one or more steps in the insulin-signaling pathway. However, there are conflicting animal data regarding whether resistin and/or RELM production are increased or decreased in obesity, and increased or decreased by thiazolidinediones, drugs known to reduce insulin resistance. The function of RELM may be primarily unrelated to their ability to promote insulin resistance. This is suggested by preliminary studies showing that RELMs inhibit adipocyte differentiation and neuronal cell survival. RELMs are also linked with inflammatory processes.

Compositions and methods to alleviate asthma by such mechanisms are thus desirable.

### SUMMARY OF THE INVENTION

One embodiment of the invention is directed to a method to reduce an allergic response in a patient by regulating expression of resistin-like molecules (RELM) alpha (RELMα) and beta (RELMβ). This may alleviate symptoms of asthma in, for example, the respiratory tract, lung, trachea, and/or lung fluid (bronchoalveolar lavage fluid), or alleviate allergic symptoms in, for example, skin, eyes, nose, throat, and/or gut.

Another embodiment of the invention is a pharmaceutical composition containing an effector of RELMα and/or β expression in a formulation and an amount sufficient to regulate DNA encoding RELMα and/or β, mRNA encoding RELMα and/or β, and/or the RELMα and/or β protein produced. The effector may be an inhibitor of STAT6 and/or an inhibitor of a Th2 cytokine, such as interleukin (IL)-4 or IL-13. The inhibitors may be small molecule inhibitors, oligonucleotide inhibitors, and/or transcriptional inhibitors.

Another embodiment of the invention is a physiological assessment method whereby patient levels of RELMα and/or β are determined, thereby providing an assessment of the patient's pulmonary status. RELMα and/or β may be determined in lung fluid, lung biopsy specimens, sputum, mucus, nasal washings, and/or blood. The specimen is analyzed so that RELMα and/or β DNA, mRNA, and/or protein is determined. As one example, Southern, Northern, or Western blots may be performed on biopsy specimens and treated with a probe to determine DNA, RNA, and protein, respectively. As another example, tissue may be appropriately stained and examined microscopically. Such methods are known to one skilled in the art. An increased level of RELMα and/or β would indicate an inflammatory process and/or a chronic repair process.

Another embodiment of the invention is a prophylactic or therapeutic method by providing RELMα and/or β in a pharmaceutically acceptable composition to the lung. The method may reduce lung acidity to treat lung inflammation, and/or may enhance epithelial repair in the lung to treat lung inflammation.

Another embodiment of the invention is a treatment method for an allergic patient. The patient is administered an amount and formulation of a pharmaceutical composition containing at least one compound capable of differentially regulating an allergen-induced gene in a patient. The compound may affect STAT6 as an anti-sense compound, a small molecule inhibitor, or a transcription inhibitor.

Another embodiment of the invention is a method to regulate insulin resistance by regulating the expression of RELMα and/or β, for example, in an adipose (fat) cell.

Another embodiment of the invention is a method to mitigate complications of obesity, such as pulmonary complications, by regulating expression of RELMα and/or β.

These and other advantages will be apparent in light of the following figures and detailed description.

### BRIEF DESCRIPTION OF THE DRAWINGS

FIG. 1 illustrates expression of RELMβ by microarray analysis during induction of experimental asthma in mice.

FIG. 2 shows Northern blots and ethidium bromide stained RNA gels demonstrating RELMA and RELMβ expression.

FIG. 3 shows Northern blots and ethidium bromide stained RNA gels demonstrating STAT6 dependence on induction of RELMs by IL4, IL-13, and allergens.

FIG. 4 shows the effect of intratracheal RELM administration on leukocyte accumulation in bronchoalveolar lavage fluid and lung.

FIG. 5 shows the effect of intratracheal RELM administration on lung histology.

FIG. 6 shows the effect of intratracheal RELM administration on in-situ cell proliferation.

FIG. 7 shows the effect of intratracheal RELM administration on collagen deposition.

FIG. 8 shows the effect or intratracheal RELM administration on fibroblast motogenic activity.

FIG. 9 shows RELM binding to a specific fibroblast receptor.

### DETAILED DESCRIPTION

RELMα and RELMβ were strongly induced in the lung by diverse allergens and by Th2 cytokines interleukin-4 (IL4) and IL-13 via a mechanism that was dependent upon the protein STAT6 (signal-transducer-and-activator-of-transcription). Both RELMα and RELMβ induced leukocyte accumulation (most prominently involving macrophages) and goblet cell hyperplasia in a dose dependent manner when delivered to the lung of naive mice. Both RELMα and RELMβ induced massive collagen deposition. In vitro, both RELMα and RELMβ had potent fibroblast motogenic activity mediated by a specific RELM receptor. These results identify the RELMs as a new family of TH2-associated cytokines with potent inflammatory and remodeling activity.

RELMα and RELMβ are members of a structurally related group of cytokines that have been associated with the resistance to insulin, and thus are associated with obesity. Obesity may be associated with a number of pulmonary abnormalities. The induction of RELMα and RELMβ by respiratory allergens such as ovalbumin and Aspergillus fumigatus, and by the Th2 cytokines IL-4 and IL-13, linked the pathogenesis of insulin resistence (obesity) and asthma.

The level of mRNA for RELMα and RELM-β was evaluated in lung from mice challenged with different allergens in different models of allergen-induced asthma.

Whole lung RNA was analyzed by DNA microarray hybridization. RNA was extracted using the Trizol (Invitrogen, Carlsbad Calif.) reagent as per the manufacturer's instructions. Following Trizol purification, RNA was repurified with phenol-chloroform extraction and ethanol precipitation.

Microarray hybridization was performed by the Affymetrix Gene Chip Core facility at Cincinnati Children's Hospital Medical Center. Briefly, RNA quality was first assessed using the Agilent bioanalyzer (Agilent Technologies, Palo Alto Calif.) and only those samples with 28S/18S ratios between 1.3 and 2 were subsequently used. RNA was converted to cDNA with Superscript choice for cDNA synthesis (Invitrogen, Carlsbad Calif.) and subsequently converted to biotinylated cRNA with Enzo High Yield RNA Transcript labeling kit (Enzo Diagnostics, Farmingdale N.Y.). After hybridization to the murine U74Av2 GeneChip (Affymetrix, Santa Clara Calif.), the gene chips were automatically washed and stained with streptavidin-phycoerythrin using a Fluidics System. The chips were scanned with a Hewlett Packard GeneArray Scanner. This analysis was performed with one mouse per chip (n≧3 for each allergen challenge condition and n≧2 for each saline challenge condition).

For Northern blot analysis, RNA was extracted from the lungs of wild-type Balb/c mice, IL-4 Clara cell 10 lung transgenic mice as described by Rankin et al., Proc. Natl. Acad. Sci USA 93:7821-5 (1996), which is expressly incorporated by reference herein in its entirety. The mice contained wild-type or deleted copies of the gene for STAT6. RNA was also extracted from the lungs of mice treated with saline or recombinant murine IL-13, as described by Pope et al., J. Allergy Clin. Immunol. 108:594-601 (2001), and by Zimmermann et al., J. Immunol. 165:5839-46 (2000), each of which is expressly incorporated by reference herein in its entirety. Hybridization was performed with ³²P-labeled cDNA encoding the sequence-confirmed murine TFF2 (I.M.A.G.E. 438574) or TFF3 (I.M.A.G.E. 1166710), obtained from American Type Culture Collection, Rockville Md.

From data image files, gene transcript levels were determined using algorithms in the Microarray Analysis Suite Version 4 software (Affymetrix). Global scaling was performed to compare genes from chip to chip; thus, each chip was normalized to an arbitrary value (1500). Each gene is typically represented by a probe set of 16 to 20 probe pairs. Each probe pair consists of a perfect match oligonucleotide and a mismatch oligonucleotide that contains a one base mismatch at a central position. Two measures of gene expression were used: absolute call and average difference. Absolute call is a qualitative measure in which each gene is assigned a call of present, marginal or absent based on the hybridization of the RNA to the probe set. Average difference is a quantitative measure of the level of gene expression, calculated by taking the difference between mismatch and perfect match of every probe pair and averaging the differences over the entire probe set.

Differences between saline and allergen-treated mice were also determined using the GeneSpring software (Silicon Genetics, Redwood City Calif.). Data were normalized to the average of the saline-treated mice. Gene lists were created which contained genes with p<0.05 and >2-fold change (using genes that received a present call based on the hybridization signal).

Mice were experimentally induced for asthma. Balb/c mice were obtained from the National Cancer Institute (Frederick Md.) and STAT6- or IL-4Rα-deficient mice (Balb/c) were obtained from Jackson Laboratory (Bar Harbor Me.). IL-13-deficient or both IL-4 and IL-13-deficient mice were kindly provided by Dr. Andrew MacKenzie. All mice were housed under specific pathogen-free conditions.

Asthma models were induced as described by Mishra et al., J. Biol. Chem. 276:8453 (2001), which is expressly incorporated by reference herein in its entirety. Briefly, ovalbumin-induced asthma was induced by intraperitoneal injection with OVA and 1 mg aluminum hydroxide (alum) separated by two weeks; followed by two doses of intranasal OVA or saline challenge two weeks later. Aspergillus fumigatus antigen-induced asthma was induced over the course of three weeks by repeated intranasal inoculation of antigen.

More specifically, mice were sensitized by i.p. injection with 100 μg OVA and 1 mg aluminum hydroxide (alum) in saline on days 0 and 14. On days 24 and 27, mice were lightly anesthetized with inhaled isofluorane and challenged intranasally (i.n.) with 50 pg OVA or saline. In other experiments, mice were challenged with nine doses of intranasal Aspergillus fumigatus antigen over the course of three weeks. Allergen (50 μl) was applied to the nares using a micropipette with the mouse held in a supine position. After instillation, mice were held upright until alert. Mice were sacrificed 18 hours following allergen challenge.

For intratracheal administration of agents, mice (22-25 gm) were anesthetized by i.p. injection (500 pg Ketaject (Ketamine HCI, Phoenix Pharmaceutical, Inc., St. Joseph Mo.). Anesthetized mice were placed upright at a 60° angle on a vertical platform. Using a flat forcep, the tongue was gently extended and a long-loading pipette tip was directly inserted into the trachea, followed by delivery of 20 μl recombinant murine IL-4 in conjunction with monoclonal antibody (10 μg) directed against IL-4 (reagents kindly provided by Dr. Fred Finkelman, University of Cincinnati); this allowed the half life of IL-4 to be increased from several minutes to about 24 hours. Dosing was every other day for six doses. Recombinant murine IL-13 (4 μg in 20 μl 0.9% saline, a generous gift from Dr. Debra Donaldson, Wyeth Research) was administered via intratracheal delivery in anesthetized mice. Dosing was for five consecutive days. Recombinant murine RELM-α and -β (10 μg in 20 μl 0.9% saline, a generous gift from PeproTech, Rocky Hill N.J.) was administered via intratracheal delivery in anesthetized mice in alternate days over a two week period.

For Northern analysis, RNA was extracted from lung tissue using Trizol reagent (Gibco-BRL, Grand Island N.Y.) following the manufacturer's protocol. Twenty micrograms of total RNA from each sample were separated by electrophoresis on 1.5% fomaldehyde agarose gels and transferred to GeneScreen hybridization membrane (Life Sciences Product, New England Nuclear, Boston Mass.) with 10×SSC (saline sodium citrate). The membrane was crosslinked by ultraviolet radiation and prehybridized at 42° C. for one hour in a 50% formamide buffer (pH 7.5) containing 10% dextran sulfate, 5×SSC, 1×Denhardt's solution, 1% SDS, 100 μg/ml herring sperm DNA, and 20 mM Tris. The ³²P-labeled cDNA probes were prepared for mouse RELMα and RELMβ using methods known to one skilled in the art and hybridized overnight at 42° C. using 1-2×10⁶ dpm/ml of the respective probes. The membranes were washed 20 min at 42° C., 20 min at 50° C., 20 min at 60° C. in 2×SSC-0.1% SDS and 20 min in 0.1×SSC-0.1% SDS. RNA isolated from 5-6 different animals was used for each experimental group.

For bronchoalveolar lavage fluid (BALF) collection, mice were euthanized by CO₂ inhalation. Immediately thereafter, a midline neck incision was made and the trachea was cannulated. The lungs were lavaged three times with 1.0 ml phosphate buffered saline (PBS) containing 1% fetal calf serum (FCS) and 0.5 mM ethylenediaminetetraacetic acid (EDTA). The recovered BALF was centrifuged at 400×g for 5 minutes at 4° C., and resuspended in 200 μl PBS containing 1% FCS and 0.5 mM EDTA. Red blood cells were lysed using RBC lysis buffer (Sigma, St. Louis Mo.) according to the manufacturer's recommendations. Total cell numbers were counted with a hemacytometer. Cytospin preparations of 5×10⁴ cells were stained with Giemsa-Diff-Quick (Dade Diagnostics of P.R., Inc., Aguada PR) and differential cell counts were determined.

For goblet cell analysis, lung tissue samples were fixed with 4% paraformaldehyde in phosphate buffer pH 7.4, embedded in paraffin, cut into 5 μm sections, and fixed to positively charge slides. Periodic Acid Schiff reaction staining (Poly Scientific R&D Corp., Bay Shore N.Y.) was then performed on the tissue sections according to the manufacturer's recommendations. Lung sections were taken from the same position in each set of mice and at least 4-5 random sections/mouse were analyzed. Using light microscopy, tissue regions associated with the entire bronchial region in the lung were quantified for percent of total mucus-producing cells relative to total number of epithelial cells.

To analyze epithelial cell proliferation, 5′-bromodeoxyuridine (BrdU) (Zymed Laboratories, San Francisco Calif.) incorporation analysis was performed. In brief, saline, RELM-α, and -β treated mice were injected i.p. with 0.25 ml 5′-BrdU (0.75 μg) three hours before sacrifice. Lung tissue was fixed with 10% neutral buffered formalin (Sigma, St. Louis Mo.) for 24 hours. After fixation, the tissue was embedded in paraffin and 5 micron sections were processed using standard histological methods. Tissues were digested with trypsin (0.125%) for three minutes at 37° C. followed by incubation for 30 minutes at room temperature. Sections were washed with PBS three times for two minutes and further incubated with monoclonal biotinylated anti-BrdU antibody for 60 minutes at room temperature. Negative controls replaced the primary antibody with PBS; positive controls were provided by the manufacturer. BrdU nuclear incorporated positive cells were detected with streptavidin-peroxidase and DAB substrate (Zymed Laboratories, San Francisco Calif.), followed by counter-staining with hematoxylin.

BrdU⁺ cell quantitation was performed with the assistance of digital morphometry by morphometric analysis using the Metamorph Imaging System (Universal Imaging Corporation, West Chester Pa.). Details of these methods are known to one skilled in the art.

For collagen staining, samples of lung tissue were fixed with 4% paraformaldehyde in phosphate buffer pH 7.4, embedded in paraffin, cut into 5 μm sections, and fixed to positively charge slides. The sections were stained with Masson's trichrome (Poly Scientific R&D Corp.) according to the manufacturer's recommendations. Collagen was quantified by morphometric analysis using the Metamorph Imaging System, as described. Lung sections were taken from the same position in each set of mice and at least 4-5 random sections/mouse were analyzed. Using digital image capture, tissue regions associated with the entire perivascular or peribrocheal region in the lung were quantified for the total collagen stained region, relative to the total tissue area. Calculated collagen levels were expressed as collagen/mm² tissue area.

The NIH 3T3 cell lines were grown in Dulbecco's modified Eagle's medium (DMEM, GIBCO BRL, Grand Island N.Y.) supplemented with 10% FCS, 50 U/ml penicillin G, 50 μg/ml streptomycin sulfate (penicillin-streptomycin, GIBCO BRL). Primary normal human lung fibroblast (NHLF) were grown in fibroblast basal medium (Clonetics-BioWhittaker, Walkersville Md.) at 37° C. and 5% CO₂-95% air. Fibroblast basal media was supplemented with 2% fetal bovine serum, human fibroblast growth factor-B (1 μg/ml), insulin (5 mg/ml), gentamicin, and amphotericin B.

Cell proliferation was assessed by the MTS (3-(4,5-dimethylthiazol-2-yl)-5-(3-carboxymethoxyphenyl)-2-(4-sulfophenyl)-2H-tetrazolium) tetrazolium assay (cell Titer96 Aqueous, Promega, Madison Wis.), which measured the number of viable cells. Between 2×10³-10⁴ cells were washed twice and plated in triplicate into microtiter-plate wells in 100 μl DMEM plus various RELM doses. Control wells containing the same concentration of reagents without cells were set up in parallel. A standard curve using a different cell number was used as a positive control. MTS (20 μl) was added to each well. Two hours after adding MTS, the plates were read in a microplate auto reader (Dynex Technologies, Billingshurst UK) at 490 nm. The result was expressed as mean optical density (OD) of the 3-well set for each dose of RELM. All of the experiments were repeated at least three times. Cell viability was also tested by trypan blue exclusion.

To monitor in vitro cell migration, twenty-four transwell units of 5-micron porosity polycarbonate filters coated with 1% gelatin (Corning Inc., Corning N.Y.) were used. 3T3 cells (5×10₅) cells/well in Hank's balanced salt solution (HBSS) (Life Technologies), pH 7.2, were placed in the upper chamber with different concentrations (10 nM to 500 nM) of RELM-α, or RELM-β in HBSS, added to the upper and lower chambers. TGF β was added to the lower chamber as a positive control. The transwell unit was kept for two hours at 37° C. humidified 95% air, 5% CO₂ atmosphere. After two hours, trypsin (0.05% with 0.53 nM EDTA, Invitrogen Corp., N.Y.) was added to the upper chamber to release adherent cells below the chamber. The media from the lower chamber was then centrifuged at 250×g, resuspended in 0.1 ml PBS, and the number of migrated cells was quantitated in the lower chamber. Each assay was set up in duplicate and repeated at least four times.

Conjugation of AlexaFluor488 (Molecular Probes, Eugene Oreg.) with RELMα was performed as recommended by the manufacturer's protocol. In brief, AlexaFluor488 was incubated with RELMα in sodium bicarbonate buffer pH 8.2 for one hour at room temperature in the dark. Free AlexaFluor was removed from the conjugated protein by gel filtration through a Coolum 1800 column (Pierce, Rockford Ill.). Specific activity was calculated by the mean fluorescence reading of AlexaFluor bound protein using excitation at 495 nm and emission at 519 nm. The specific activity was 0.726 MF/μg.

AlexaFluor conjugated RELM-α binding to 3T3 cells was determined by flow cytometric analysis using FACScan (Becton Dickinson, San Jose Calif.). The 3T3 cells were incubated with different concentrations of AlexaFluor488 conjugated RELMα (50 ng/ml to 1000 ng/ml) in the presence or absence of unlabeled RELMα for one hour on ice. The cells were washed with PBS three times for five minutes at 4° C. Binding with AlexaFluor488 conjugated bovine serum albumin (BSA) was used as a negative control.

AlexaFluor conjugated RELMα ligand binding to 3T3 cells was determined by fluorocytometer with a maximum excitation wavelength of 495 nm and an emission wavelength of 519 nm. 3T3 cells were incubated with different concentrations of AlexaFluor488-conjugated RELMα (1 nM to 160 nM) for one hour on ice. Cells were washed with sodium bicarbonate buffer, pH 8.3, three times for five minutes at 4° C. The cell free supernatants were pooled for measuring the free protein concentration. The cells were resuspended in sodium bicarbonate buffer, pH 8.3, and bound protein levels were measured by fluorescence. A standard curve of AlexaFluor488 conjugated RELMα was prepared to calculate free and bound RELMα.

Airway reactivity to methacholine was assessed in conscious, unrestrained mice by barometric plethysmography, using apparatus and software supplied by Buxco (Troy N.Y.). This system yields a dimensionless parameter known as enhanced pause (Penh), reflecting changes in wave form of the pressure signal from the plethysmography chamber combined with a timing comparison of early and late expiration, which can be used to empirically monitor airway function.

Mice treated with saline or one of seven doses of RELMα or RELMβ were placed in the chamber, and baseline readings were taken and averaged for three minutes. Aerosolized methacholine (concentration in solution ranging from 3.125 mg/ml to 50 mg/ml) was then delivered through an inlet into the chamber for two minutes. Readings were averaged over three minutes after each dose was administered. As a control, airway resistance to methacholine after the administration of 10 μg IL-13 was also determined following a similar protocol.

All data are expressed as mean ± Standard Deviation (SD). Statistical significance comparing different sets of mice was determined by the Student t-test.

RNA obtained from saline- and allergen-challenged mice was subjected to microarray analysis utilizing the Affymetrix chip U74Av2. This chip contains oligonucleotide probe sets representing 12,423 genetic elements, one of the largest collection of characterized mouse genes commercially available. Allergen-challenged mice (OVA or Aspergillus) were compared to their respective saline control (n=3-6 mice in each experimental group) and genes which showed at least a two-fold statistically significant increase (p<0.05) following allergen challenge were identified.

Compared with mice challenged with saline, OVA-challenged mice had 496 genes induced and Aspergillus fumigatus-challenged mice had 527 genes induced. The majority (59% of OVA and 55% of Aspergillus) of the induced transcripts overlapped between the two experimental asthma models.

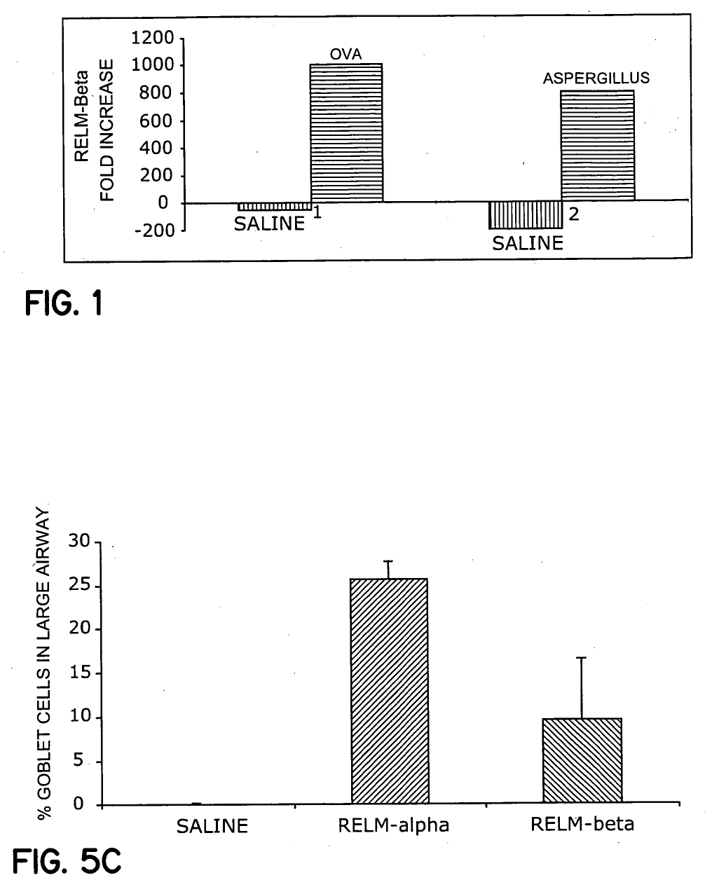

With reference to FIG. 1, global quantitative microarray analysis revealed significantly increased expression of RELMβ mRNA during allergen induced asthma in each of the two distinct models, relative to control mice. The expression of RELMβ in the lungs of mice challenged with OVA indicated a 985-fold increase. The expression of RELMβ in the lungs of mice challenged with Aspergillus indicated a 600-fold increase.

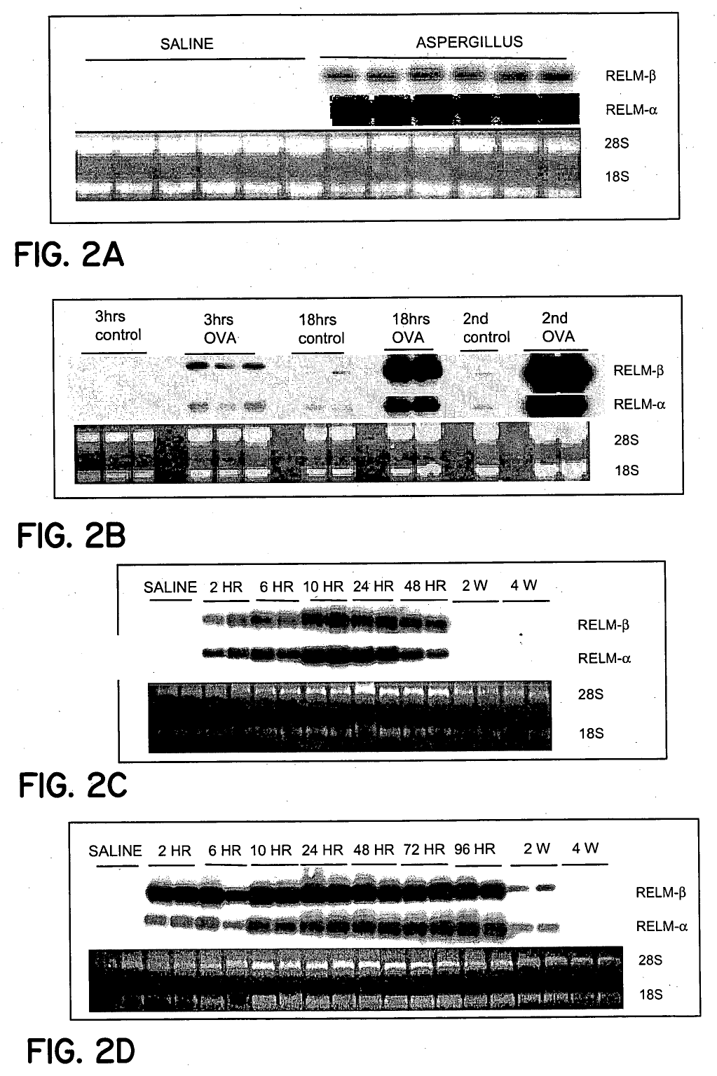

With reference to FIG. 2, Northern blot analysis confirmed that the expression of RELMβ was induced in the lungs of mice challenged in both OVA- and Aspergillus-induced models. Northern blots of lung RNA demonstrated up-regulation of RELMβ mRNA in the wild type mice induced with the Aspergillus (FIG. 2A) or OVA (FIG. 2B) allergen, compared to lungs of wild type control mice administered a saline challenge. RNA standards (28S and 18S) are indicated.

To determine if induction if RELMβ was specific for this family member, the same Northern blots were probed with RELMβ and resistin cDNA probes; the microarray chip did not contain sequences that encoded RELMα and resistin. RELMα but not resistin was markedly induced by both allergen challenges (FIGS. 2A and 2B, and data not shown). OVA-induced RELMα and RELMβ expression was time and dose dependent during the progression of experimental asthma. FIG. 2B demonstrates that RELM mRNA was induced after the first allergen challenge, and induced even higher following two OVA allergen challenges.

The induction and decline of RELM mRNA accumulation in the lung following the first and second OVA challenge was examined. Following the first OVA challenge, RELMα and RELMβ mRNA both peaked between 6 to 10 hours, and declined to baseline by two weeks. Following two allergen challenges, RELM mRNA accumulated at a much higher level, reaching near peak values by only two hours and remaining elevated even after 96 hours, compared to saline challenged lungs, but returning to baseline after four weeks (FIGS. 2C and 2D).

RELM mRNA accumulation was correlated with differential leukocyte recruitment into the BALF. Following both OVA challenges, peak RELM mRNA expression correlated with total BALF cell levels. However, the sustained RELM expression more strongly correlated with the number of macrophages and eosinophils that were increased between 10 to 96 hours, and returned to baseline after 2 weeks (data not shown). Both RELMα and RELMβ had similar kinetic expression patterns.

Asthma is a Th-2 associated process. Thus, the effect of pharmacological delivery of IL-4 and IL-13 on induction of RELMs was evaluated. The cytokines IL-4 or IL-13 were repeatedly applied to the respiratory tract of anesthetized mice. This protocol produces several features of experimental asthma including eosinophilic inflammation, chemokine induction, mucus production, and AHR.

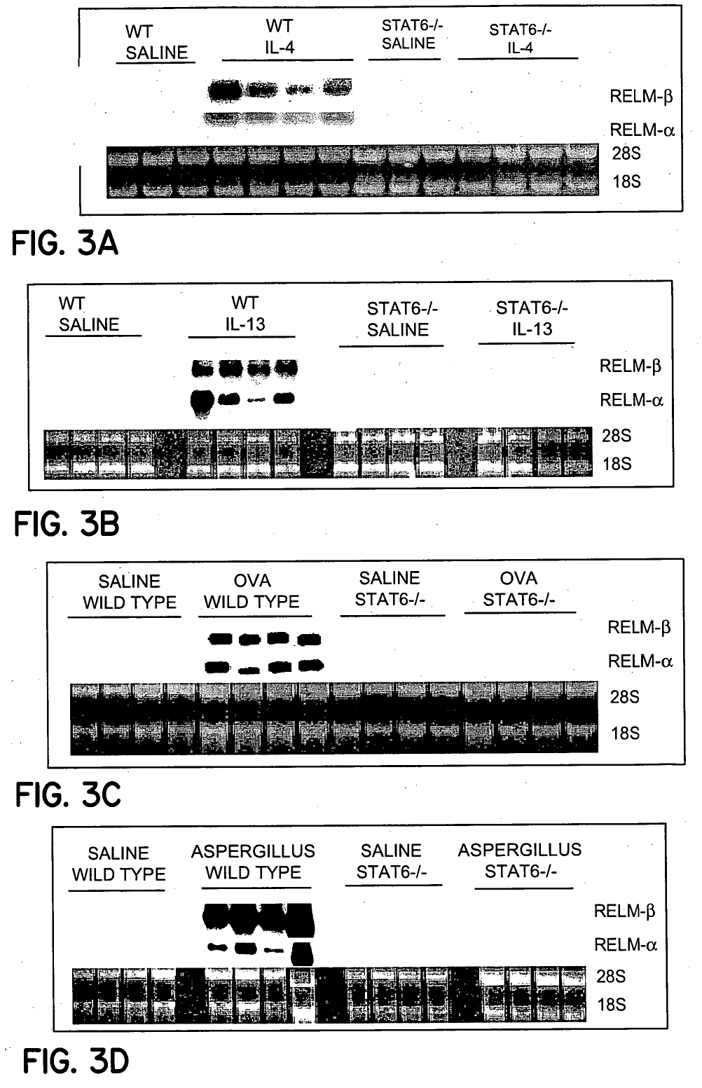

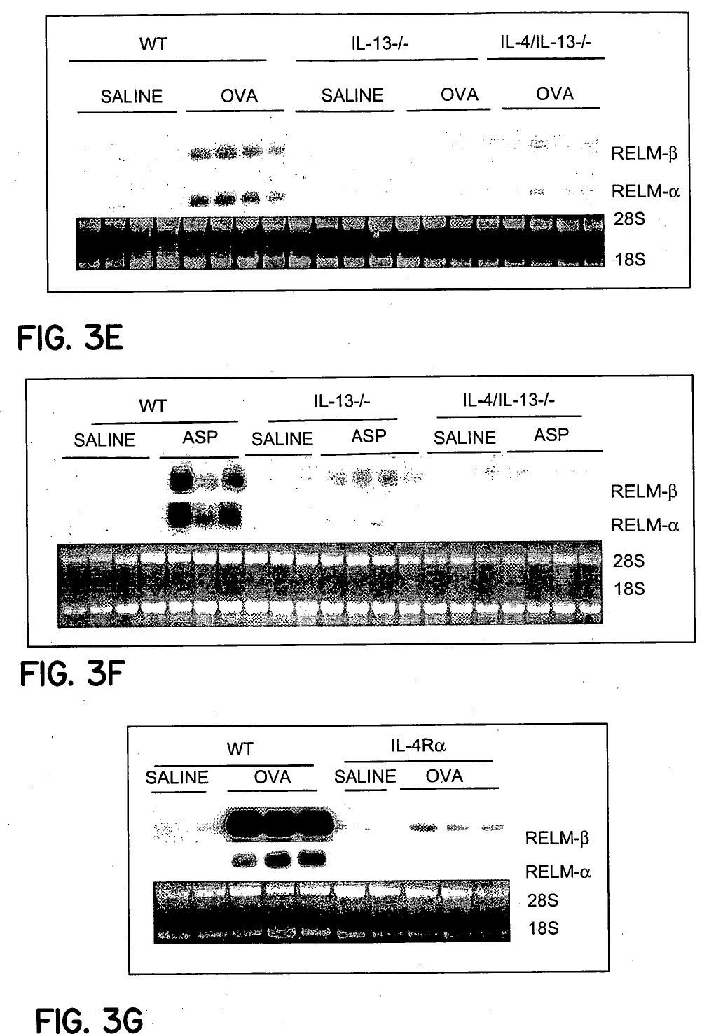

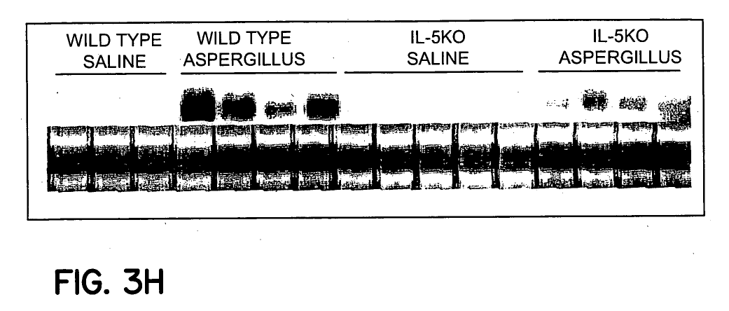

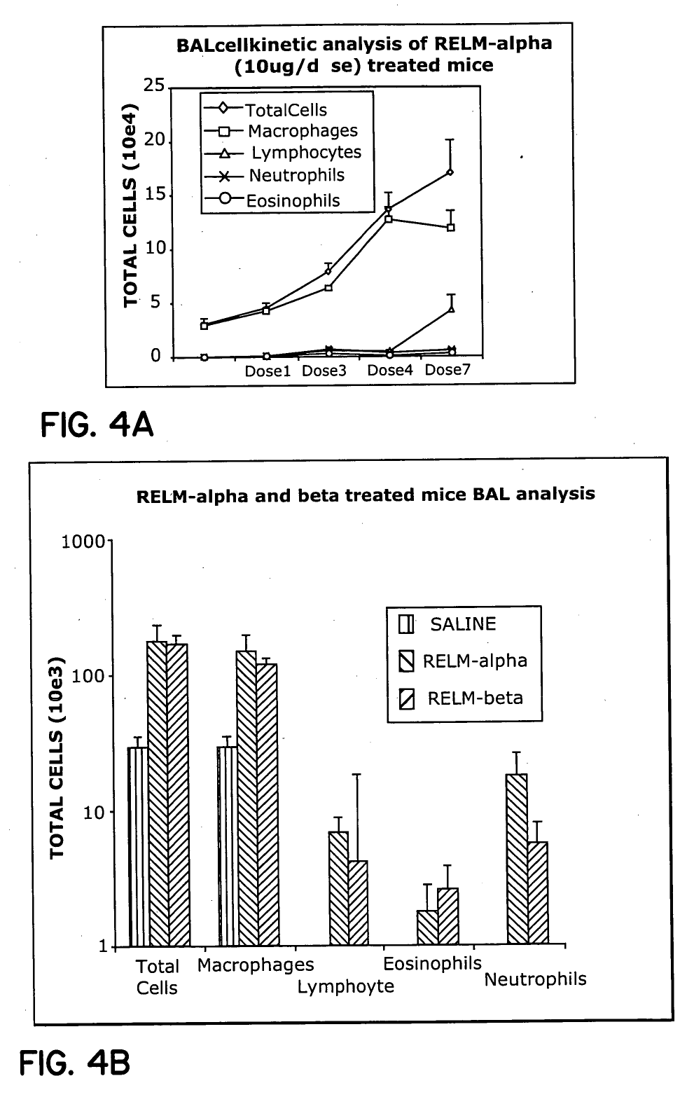

Administration of both TH-2 cytokines induced marked levels of RELM mRNA compared with saline treated control mice (FIGS. 3A and 3B). To test the role of STAT6 in the induction of RELM in vivo-+4 was delivered to wild-type and STAT6-deficient mice. As shown in FIG. 3A, IL-4-induced RELM expression was largely STAT6 dependent. IL-13 was also administered to STAT6 deficient mice and, as shown in FIG. 3B, RELM expression was also largely STAT6 dependent.

If allergen-induced RELM mRNA expression was STAT6 dependent, it would help determine if allergen-induced RELM was predominantly downstream from IL-4/IL-13 signaling. As shown in FIG. 3C, mice deficient in STAT6 had no expression of RELMs following OVA challenge, compared with OVA-challenged wild-type mice. Similarly, Aspergillus-induced RELM was largely STAT6 dependent (FIG. 3D).

To further examine the role of IL-4 and IL-13 in allergen-induced RELM expression, OVA and Aspergillus-induced experimental asthma in mice genetically deficient in IL-13, or double deficient in IL-4 and IL-13, were assessed. IL-13 gene targeted mice had barely detectable induction of RELMs mRNA (FIGS. 3E and 3F). Mice deficient in both IL-4 and IL-13 were completely resistant to Aspergillus- and OVA-induced RELM mRNA accumulation. Because activation of STAT6 signaling by IL-4 and IL-13 is usually mediated by IL-4Rα chain, RELM induction in OVA-challenged mice that lack functional IL-4Rα was examined. As shown in FIG. 3G, RELM induction was significantly attenuated in IL-4Rα-deficient mice, as compared to wild type mice.

FIG. 3H shows Northern blots and ethidium bromide stained RNA gels from the lung of mice that were either wild type or IL-5-deficient and challenged with Aspergillus fumigatus. IL-5 is a strong inducer of eosinophils, cells which are increased in an allergic inflammatory response. As shown in FIG. 3H, both wild type and IL-5 deficient (knockout) mice expressed similar amount of RELMβ mRNA.

Collectively, these data established that RELMα and RELMβ were Th-2 associated cytokines. In particular, RELMα and RELMβ were induced by IL-4 and IL-13, and allergy-induced induction of RELM was largely predicted by IL-13.

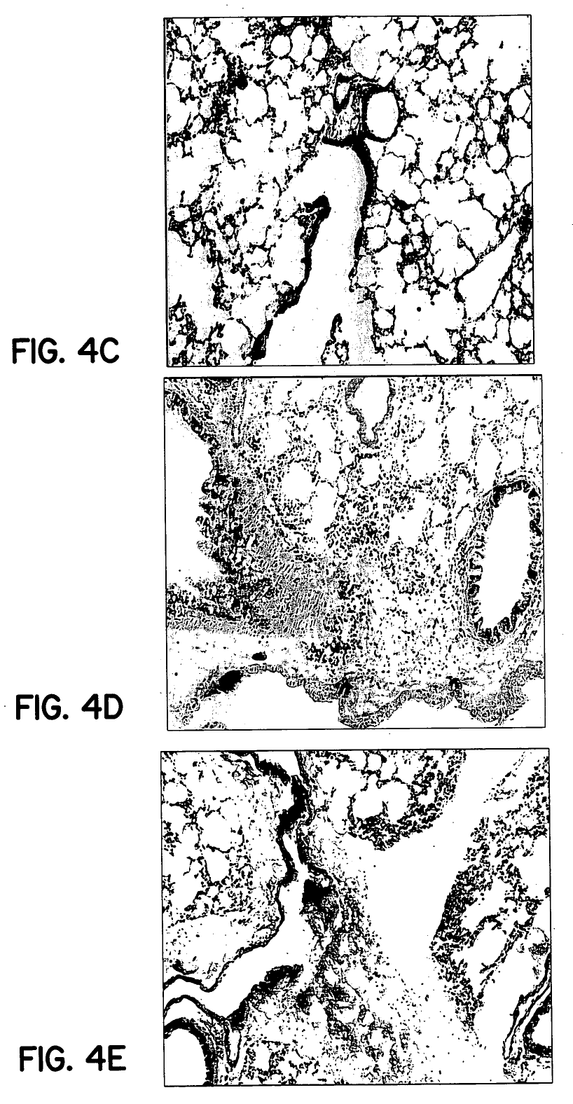

To determine whether over-expression of RELMs in the lung would induce at least a partial asthma-like phenotype, recombinant murine RELMα and RELMβ were administered via intratracheal delivery, and the effect on the level of cells in BALF was examined. Repeated doses of RELMα (10 μg) were administered, and quantitative analysis of BALF cells was performed 18 hours following each dose. As shown in FIG. 4A, RELMα induced substantial increases in total BALF cells. Differential analysis revealed that macrophages and lymphocytes were the predominant cell type affected. Following seven doses of intratracheal RELMα, there was about a 6-fold increase in the total cell count as well as total number of macrophages (FIG. 4B). As a control, heat treated (15 minutes) RELMα protein was completely inactivated (data not shown), indicating that proper protein folding was required for RELM activity. Representative histological sections of the lung following treatment with control saline (FIG. 4C), RELMα (FIG. 4D) and RELMβ (FIG. 4E) revealed the presence of perivascular and peribronchial inflammatory cell infiltrates.

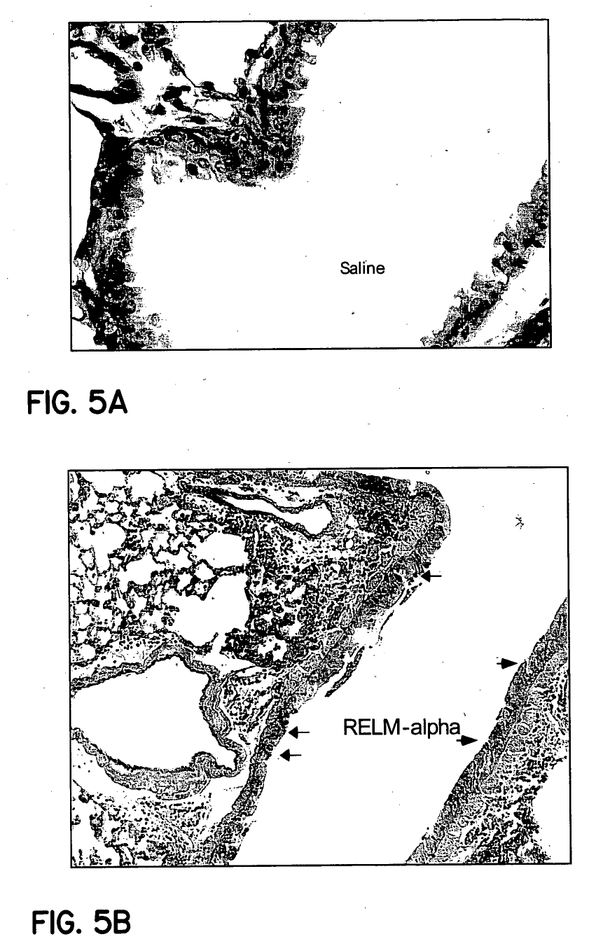

After intratracheal administration of RELM, lung histology revealed apparent epithelial metaplasia, suggesting goblet cell differentiation (FIGS. 5A and 5B). Lung sections were stained for mucus production with Periodic Acid Schiff (PAS), revealing a large increase in mucus PAS positive (PAS+) cells following RELMα or RELMβ administration, compared with saline administration (FIG. 5C). Quantitative analysis of PAS+ cells revealed 25.6±8.7 and 9.4±6.9 percent goblet cells in relation to large airway epithelial cells following RELMα and RELMβ administration, respectively (mean±S.D., n=3). In saline treated mice, no goblet cells were detected by PAS tissue staining.

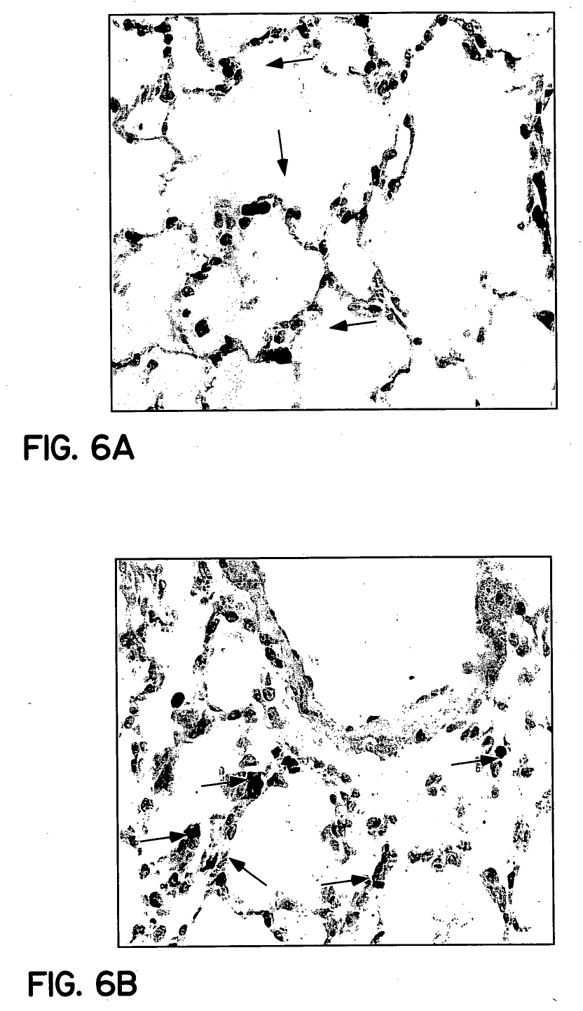

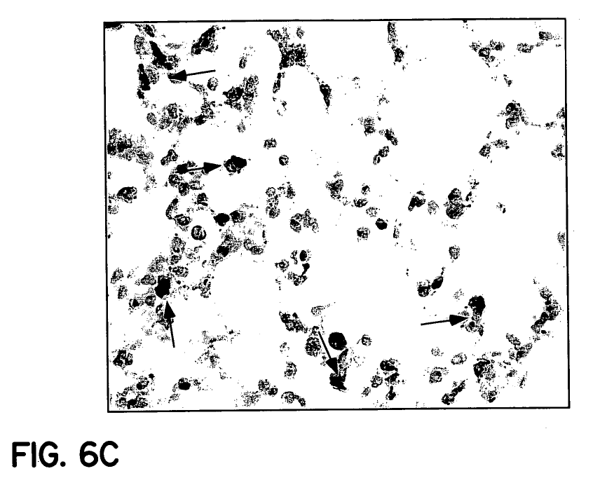

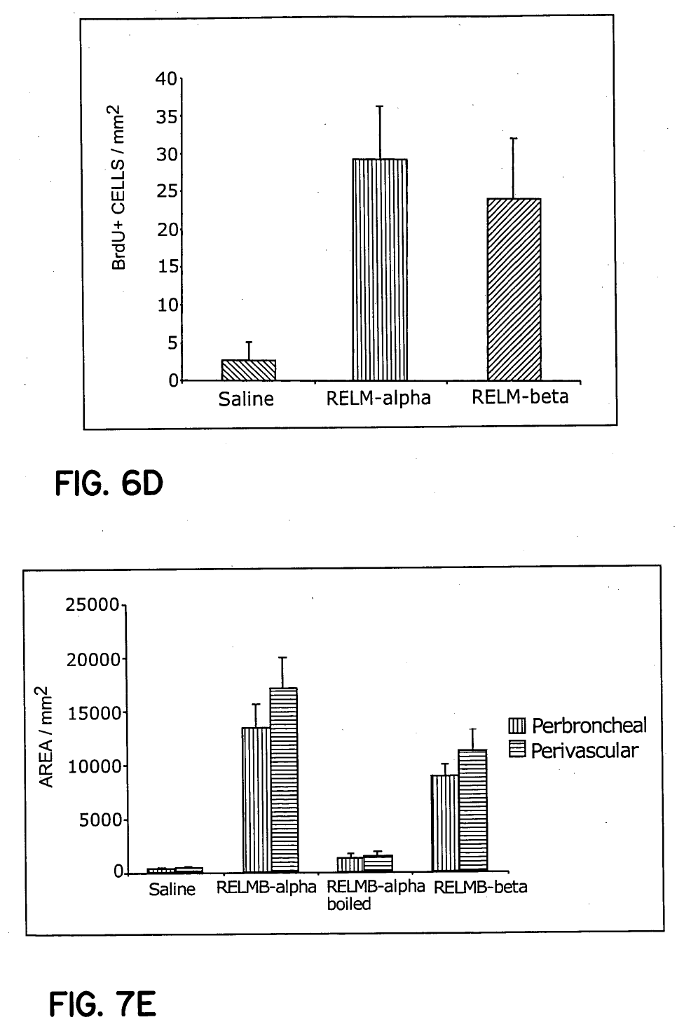

Examination of the lung histology following RELM administration revealed an abundance of cell populations with active mitosis (FIGS. 6A-C). To further examine in situ cell proliferation induced by RELMs, mice were treated with seven doses of intratracheally administered RELMs, and were subsequently administered BrdU three hours before sacrifice for analysis of BrdU incorporation into the lungs.

Analysis revealed a large increase in BrdU positive (BrdU+) cells following administration of RELMα (FIG. 6B) or RELMβ (FIG. 6C), compared with saline treated mice (FIG. 6A). Quantitative analysis of BrdU+ cells revealed 2.6±2.4, 29.22±7, and 24±7.9 cells/mm² (mean±S.D., n=3) following saline, RELMα, and RELMβ administration, respectively (FIG. 6D). Multiple cell types were induced to incorporate BrdU, including epithelial cells and inflammatory cells.

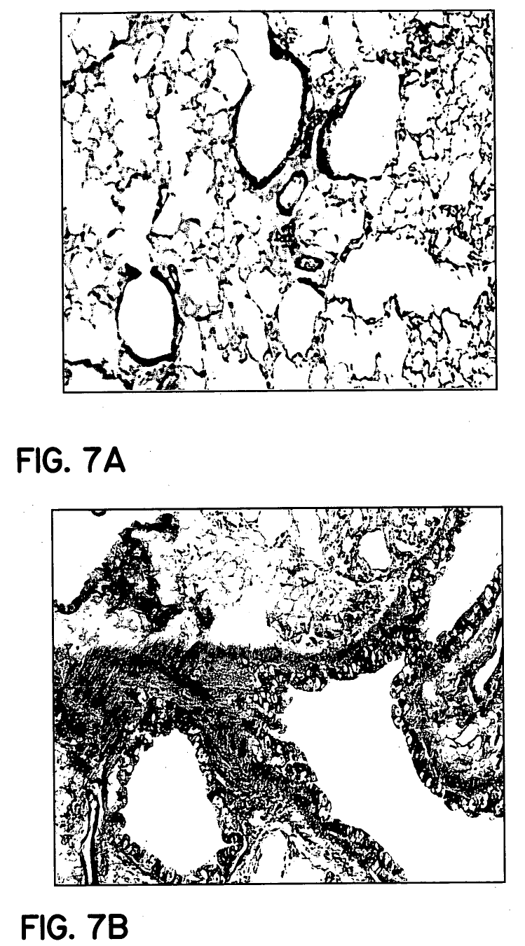

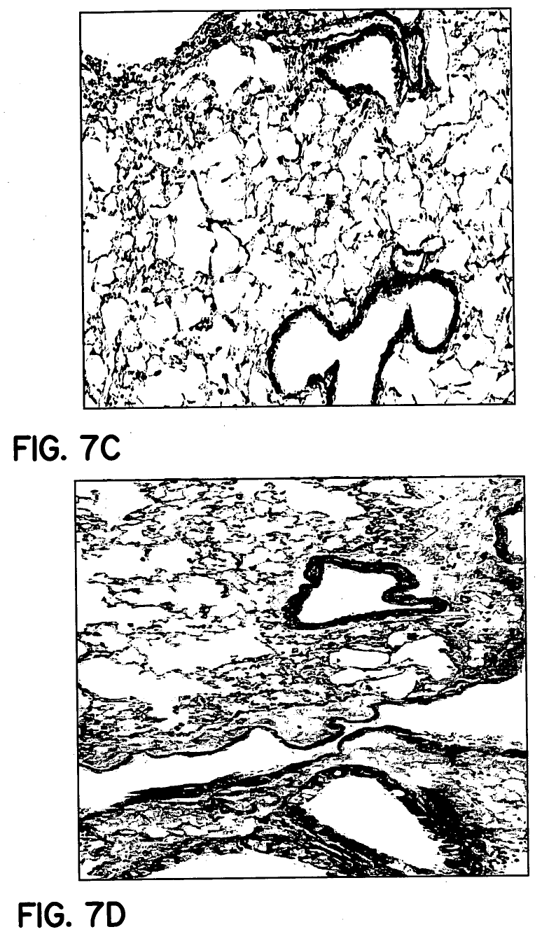

Lung histology following RELM administration revealed an apparent thickness of the reticular basement membrane, suggesting collagen deposition (FIGS. 7A-7D). Lung sections from mice treated with seven doses of intratracheally administered RELMs and stained for collagen with trichrome stain confirmed an impressive thickening of the airway reticular basement membrane composed of trichrome positive material (FIGS. 7A-7D). Quantitative analysis of the trichrome positive layer around bronchi revealed thicknesses of 355±50, 1322±429, and 8807±1126 area/mm² following saline, RELMα and RELMβ administration, respectively (FIG. 7E). Quantitative analysis of the trichrome positive layer surrounding blood vessels also revealed increased thicknesses of 462±114, 17063±2936, and 11233±1939 area/mm² (mean±S.D., n=4) following saline, RELMα and RELMβ administration, respectively (FIG. 7E). As a control, when RELMα was boiled prior to its administration, its ability to induce collagen deposition was lost (FIG. 6E).

Collagen deposition in the lungs of mice treated with RELM prompted examination of the direct effect of recombinant RELMs on fibroblasts in vitro. To determine if RELM treatment would induce fibroblast proliferation, murine 3T3 fibroblasts were exposed to a full dose range of RELMα and RELMβ for 24-72 hours and their proliferative response was measured. This exposure failed to induce 3T3 cell proliferation even though control treatment induced proliferation (data not shown).

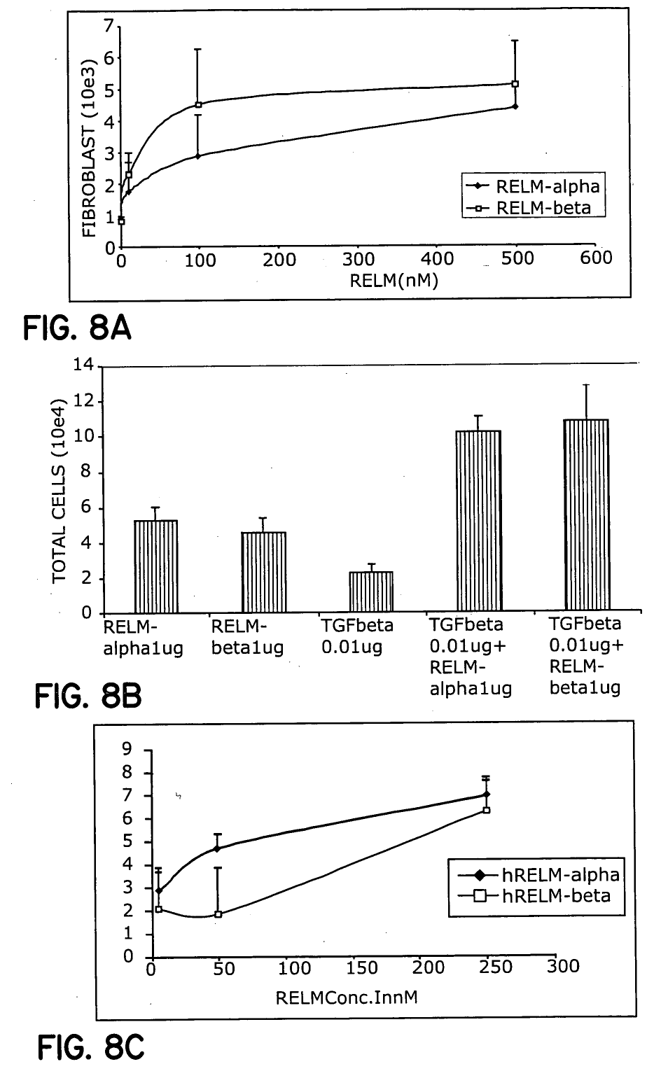

To determine whether RELMs might be mediating collagen deposition by inducing fibroblast accumulation in the lung, at least in part, the ability of RELM to induce 3T3 fibroblast chemoattraction in vitro was analyzed. Both RELMα and RELMβ induced dose-dependent 3T3 cell movement through a transwell membrane; activity was seen with doses as low as 10 nm, and a plateau was seen between about 100 nM to about 500 nM. As a positive control, exposure to TGFβ (40 mM), a known fibroblast motogen, induced 8-fold increase in fibroblast movement (data not shown). Administration of sub-optimal concentrations of both TGFβ and RELMα or RELMβ revealed that these cytokines had additive effects (FIG. 8B).

To determine if RELM promoted 3T3 movement in vitro by a chemotactic or chemokinetic mechanism, different concentrations of RELM were added above and below the membrane in the in vitro assay. This checkerboard analysis revealed that alterations in the RELM concentration gradient did not affect cell movement (data not shown); thus, RELM induced fibroblast chemokinesis rather than chemotaxis.

To determine if the ability of RELM to induce fibroblast movement in a murine system was applicable to a human system, the chemotaxis activity of human recombinant RELMα and RELMβ on human primary lung fibroblasts was examined. As shown in FIG. 8C, human RELM had similar activity on human lung fibroblasts as in the murine system.

The data suggested that RELM was a cytokine with potent activity on fibroblasts; we thus determined whether fibroblasts expressed a specific RELM receptor. There are no known RELM receptors identified to date.

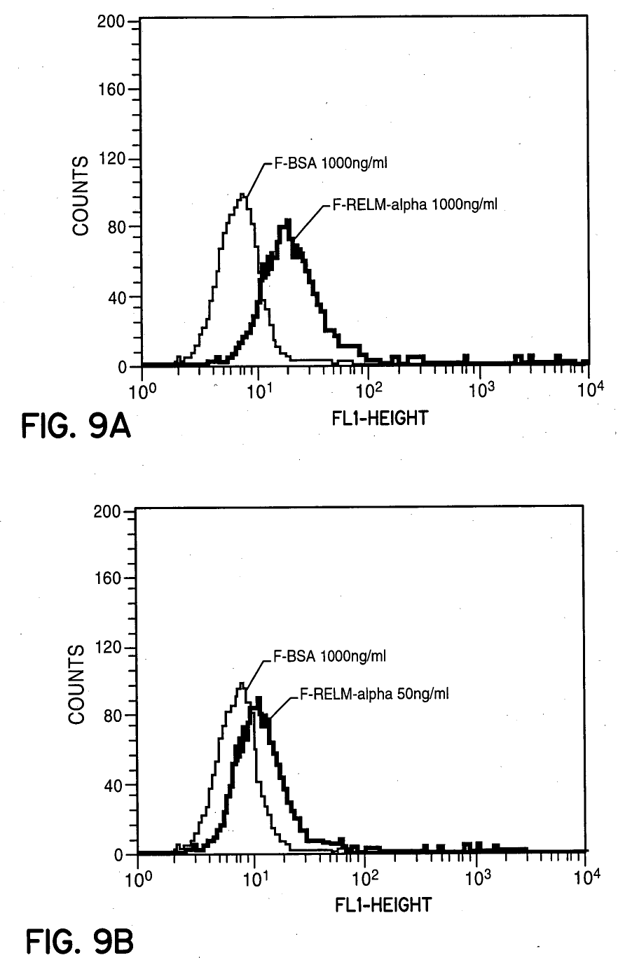

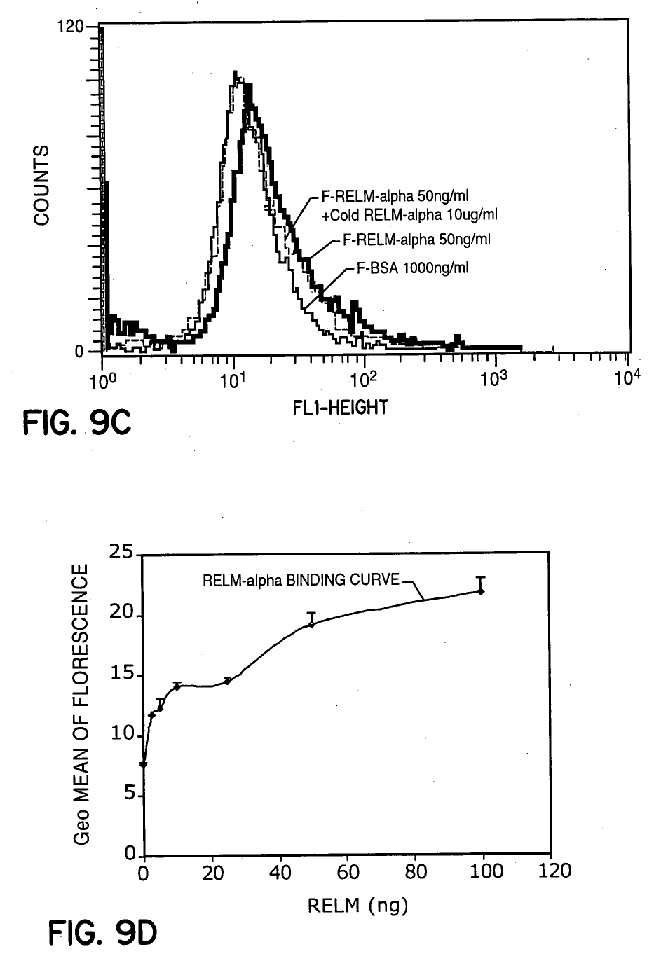

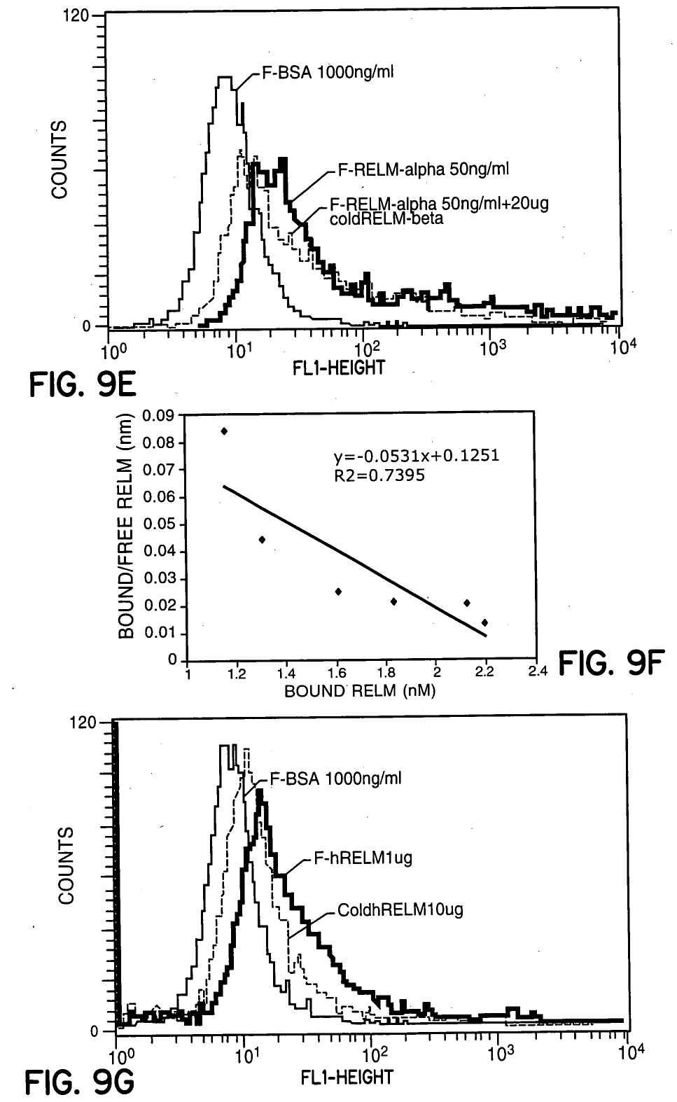

Recombinant murine RELMα was labeled with the AlexaFluor488 probes and binding to 3T3 fibroblasts was measured using FACS. As shown in FIG. 9A, RELMα displayed saturable binding to 3T3 fibroblasts. Activity was noted between 50 ng/ml and 1000 ng/ml and reached a plateau above concentrations of 1000 ng/ml (FIGS. 9A-9C). Addition of unlabeled RELMαcompletely eliminated the binding of labeled RELMα (FIG. 9D). Similarly, addition of unlabeled RELMβ reduced the binding of labeled RELMα (FIG. 9E). RELMα and RELMβ blocked the binding of each other, indicating they shared the same receptor(s). Finally, Scatchard analysis revealed that the RELMα receptors had Kd of 19.9±1.2 nM. 3T3 cells have more than one binding site for RELMα or RELM (FIG. 9F). Binding of labeled recombinant human RELMα to human lung fibroblasts was also examined. Binding properties in human lung fibroblasts with human RELM, as shown in FIG. 9G are similar to the murine system, as shown in FIG. 9C.

The ability of RELM to induce AHR was evaluated, in view of the ability of RELM to induce some of the hallmark features of allergic airway disease. Naive animals were exposed to seven doses on alternate days of RELMα, RELMβ, or control saline. Their responsiveness to a full dose of nebulized methacholine was measured 24 hours after the last dose of RELM or saline. RELM treated mice failed to develop AHR, as measured by changes in Penh (data not shown). As a control, mice treated with five doses of recombinant IL-13, developed a two-fold increase in their methacholine responsiveness (data not shown).

Genes were analyzed which were not specific to a particular experimental regimen, thus, two independent models of asthma were used. The allergen-induced genes which overlapped in these two independent models were analyzed using global transcript profile analysis. Both asthma models, however, have similar phenotypes, including Th2 associated eosinophilic inflammation, mucus production, and airway hyperresponsiveness (AHR).

In one model, mice were intraperitoneally sensitized with the allergen OVA in the presence of the adjuvant alum on two occasions separated by fourteen days. Subsequently, mice were challenged with intranasal OVA or control saline on two occasions separated by three days. Eighteen hours after the last allergen challenge, the lung was harvested for RNA analysis. In another model, experimental asthma was induced by the Aspergillus fumigatus antigen, a ubiquitous and common aeroallergen. This model involved a unique mucosal sensitization route (intranasal), compared with the OVA model. Lung RNA was obtained eighteen hours after nine doses of intranasal Aspergillus fumigatus allergen or saline challenges.

Messenger RNA (mRNA) encoding RELM-β was not detectable in lungs from mice administered a saline control. Messenger RNA encoding RELM-β was markedly increased following the development of OVA and Aspergillus fumigatus antigen-induced experimental asthma. The expression of RELM-β in the lungs of OVA-challenged mice was time and dose dependent. The expression of RELM-β was also induced in lungs from IL-4 lung transgenic mice, and in mice that were administered IL-13 by an intratracheal route.

Additionally, the transcription factor STAT6 had an effect on RELM-β expression. Mice that were deficient in STAT6 were either challenged with the OVA allergen or Aspergillus fumigatus allergen, or were administered IL-4 or IL-13. The induced RELM-beta expression was demonstrated to be dependent upon this transcription factor. In contrast, IL-5 deficient mice had normal induction of RELM-beta compared with wild type mice.

To understand the complex mechanisms involved in the pathogenesis of asthma, transcript expression profile analysis was used to define a set of “asthma signature” genes. The discovery of RELMβ as an asthma-associated gene indicated this molecule had properties potentially important in asthmatic responses. RELMβ has not been previously implicated in the pathogenesis of asthma.

Allergic lung inflammation, triggered by diverse allergens and modes of disease induction, was associated with marked and specific ectopic expression of RELMβ in the lung. This is in contrast to prior work, which established RELMβ as a member of the resistin family of proteins, a structurally related group of cytokines associated with resistance to insulin and obesity.

The Th2 cytokines IL4 and IL-13 induced RELMβ in the lung. Thus, allergen-induced RELMβ was mediated, at least in part, by IL4 and IL-13. IL-4 and IL-13 are related cytokines that share a similar signaling mechanism (e.g. utilization of a common receptor subunit (IL-4Rα chain) and activation of STAT6). Both of these cytokines were known to play roles in asthma, but the mechanisms by which they induced various elements of the asthmatic response (e.g. AHR, mucus production, and airway remodeling) were only partially understood. The present invention demonstrates that the pathogenesis of IL-4/IL-13-associated allergic lung responses is mediated by RELMβ, at least in part. Injury-associate epithelial hyperplasia and epithelial differentiation (e.g. mucus cell metaplasia), may also be mediated by RELMβ in the lung. RELMβ may also alter mucus production.

RELMβ was induced by allergens and both IL-4 and IL-13 by a mechanism which depended upon STAT6. These data were consistent with studies that have shown distinct and overlapping mechanisms for the involvement of IL-4 and IL-13 in experimental asthma (Wills-Karp, M., J. Allergy Clin. Immunol. 107:9-18 (2001)). Additionally, while OVA and Aspergillus both induce experimental asthma, Aspergillus was capable of inducing Th2 responses independent of adjuvant. This indicated that both allergens employ distinct mechanisms for asthma induction.

RELMβ is a secreted protein. It has been identified as expressed in the gastrointestinal tract, particularly the colon, with marked increases in tumors, suggesting a role in intestinal proliferation. This suggested that RELMβ may be involved in regulating epithelial proliferation in response to injury. The asthmatic lung is characterized by a large increase in epithelial proliferation. There may be a role for RELMβ in promoting mucosal healing through inhibition of acid secretion and stimulation of epithelial proliferation. Allergen-induced RELMβ may play a role in regulating several features associated with the pathogenesis of asthma, including acidification of the airway and epithelial proliferation.

The ability to utilize RELMβ as a diagnostic tool is also disclosed. Qualitative and quantitative determinations of RELMβ are markers of an inflammatory process. Thus, RELMβ determinations may be used to assess a patent's clinical status, phenotype, genotype, drug response, and/or prognosis, and single nucleotide polymorphisms. For example, an increased amount of RELMβ in pulmonary tissue obtained from a biopsy site would indicate an inflammatory process and/or a chronic repair process. Amounts of RELMβ may be assessed in, for example, lung fluid, lung biopsy specimens, sputum, mucus, nasal washings, and/or blood. The specimen is analyzed so that RELMβ DNA, mRNA, and/or protein is determined. As one example, Southern, Northern, or Western blots may be performed on biopsy specimens and treated with a probe to determine DNA, RNA, and protein, respectively. As another example, the tissue may be histologically evaluated, for example, by appropriate staining and microscopic examination. Such methods are known to one skilled in the art.

RELMβ is an allergen-induced gene in the asthmatic lung. The Th2 cytokines IL-4 and IL-13 induced expression of RELMβ. IL-5 did not induce expression of RELMβ. Induction occurred by a STAT6 dependent mechanisms. Thus, RELMβ was involved with the pathogenesis of asthma.

RELMβ involvement in the asthmatic lung included processes implicating RELMβ in the control of insulin resistance. This includes regulating the differentiation of mesenchymal cells, which are immature fibroblasts that may develop into a variety of mature cell types, such as adipocytes. Responses of the allergic lung shared pathogenic mechanisms with processes related to obesity, such as insulin resistance and diabetes, which may be associated with a number of pulmonary abnormalities.

Compositions affecting RELMβ may be small molecule inhibitors, oligonucleotide inhibitors, and/or transcriptional inhibitors of STAT6 or Th2 cytokine inhibitors. Concentrations of these inhibitors in the composition may be prepared for doses ranging from about 0.01 mg/kg to about 100 mg/kg of body weight. The amounts of inhibitors in the composition may vary depending on the type of formulation. Compositions may be administered to a mammal, such as a human, either prophylactically or in response to a specific condition or disease. For example, the composition may be administered to a patient with asthmatic symptoms and/or allergic symptoms including allergic rhinitis, asthma, and/or eczema. The composition may be administered non-systemically such as by inhalation, aerosol, drops, etc.; systemically by an enteral or parenteral route, including but not limited to intravenous injection, subcutaneous injection, intramuscular injection, intraperitoneal injection, oral administration in a solid or liquid form (tablets (chewable, dissolvable, etc.), capsules (hard or soft gel), pills, syrups, elixirs, emulsions, suspensions, etc.). As known to one skilled in the art, the composition may contain excipients, including but not limited to pharmaceutically acceptable buffers, emulsifiers, surfactants, electrolytes such as sodium chloride; enteral formulations may contain thixotropic agents, flavoring agents, and other ingredients for enhancing organoleptic qualities.

What is claimed is:

1. A method to mitigate an allergic response in a patient comprising administering to the patient a pharmaceutically acceptable formulation of a composition comprising an inhibitor of at least one of resistin-like molecule α (RELMα), resistin-like molecule β (RELMβ), RELMα receptor binding, or RELMβ receptor binding, in an amount sufficient to inhibit RELMα or RELMβ and thereby mitigate the allergic response.

2. The method of claim 1 wherein the composition is administered to at least one of an airway, lung, trachea, respiratory tract, or bronchoalveolar space.

3. The method of claim 1 wherein administration is by a route selected from the group consisting of intravenously, intranasally, intratracheally, subcutaneously, intramuscularly, orally, intraperitonally, and combinations thereof.

4. The method of claim 1 wherein the inhibitor down-regulates expression of at least one of RELMα and RELMβ.

5. The method of claim 1 wherein the inhibitor is selected from at least one of IL-4 and IL-13.

6. The method of claim 1 wherein the composition further regulates at least one of insulin resistance or obesity.

7. A pharmaceutical composition comprising an inhibitor of at least one of resistin-like molecule α (RELMα) expression or resistin-like molecule β (RELMβ) expression in a pharmaceutically acceptable formulation sufficient to inhibit at least one of an amount of DNA encoding RELMα, DNA encoding RELMβ, mRNA encoding RELMα, mRNA encoding RELMβ, RELMα protein or RELMβ protein.

8. The composition of claim 7 comprising an inhibitor of STAT6, an inhibitor of a Th2 cytokine, or combinations thereof.

9. The composition of claim 7 comprising an inhibitor selected from at least one of a small molecule inhibitor, an oligonucleotide-inhibitor, a transcriptional inhibitor, a translational inhibitor, and combinations thereof.

10. The composition of claim 7 in a formulation for administration to at least one of an airway, lung, trachea, respiratory tract, or bronchoalveolar space in an asthmatic patient.

11. A physiological assessment method comprising determining a level of at least one of resistin-like molecule α (RELMα) or resistin-like molecule β (RELMβ) in a patient to assess a patient parameter selected from the group consisting of clinical status, phenotype, genotype, drug response, prognosis, single nucleotide polymorphisms, and combinations thereof.

12. The method of claim 11 wherein RELMα or RELMβ is determined in at least one of lung fluid, lung biopsy, sputum, mucus, nasal washings, bronchoalveolar fluid, respiratory tract tissue, respiratory tract fluid, blood, and combinations thereof.

13. The method of claim 11 wherein at least one of RELMα DNA, RELMβ DNA, RELMα mRNA, RELMβ mRNA, RELMα protein, or RELMβ protein is determined.

14. The method of claim 11 wherein an increased level of at least one of RELMα or RELMβ indicates an inflammatory process.

15. The method of claim 11 wherein at least one of RELMα or RELMβ is determined qualitatively, quantitatively, or functionally.

16. A method to mitigate lung disease in a patient comprising providing an inhibitor to at least one of a resistin-like molecule α (RELMα) or resistin-like molecule β (RELMβ) in a pharmaceutically acceptable composition to a lung of a patient to thereby mitigate lung disease.

17. The method of claim 16 wherein the mitigation comprises reducing at least one of lung leukocyte accumulation, mucus production, cell proliferation, collagen deposition, macrophage accumulation, and fibroblast accumulation.

18. The method of claim 16 further comprising inhibiting expression of at least one of RELMα and RELMβ in an airway, lung, trachea, respiratory tract, or bronchoalveolar lavage fluid.

19. The method of claim 16 wherein the patient is asthmatic

20. The method of claim 16 wherein the patient has lung fibrosis.

21. The method of claim 16 wherein a pharmaceutical composition of a regulatory compound is administered to the lung in an amount sufficient to down regulate RELM.

22. The method of claim 21 wherein the composition is administered by a route selected from the group consisting of intranasal, intratracheal, aerosol, inhalation, subcutaneously, intramuscularly, orally, intraperitoneally, and combinations thereof.

23. The method of claim 16 wherein the regulation reduces expression of RELM to mitigate lung disease.

24. The method of claim 16 wherein at least one of lung fibrosis, lung inflammation, asthma, lung congestion, lung scarring, and inflammatory cell recruitment is mitigated.

25. The method of claim 24 wherein mitigation of inflammatory cell recruitment is at least one of fibroblasts, macrophages, lymphocytes, neutrophils, and eosinophils

26. A method to enhance repair of allergy-induced inflamed lung tissue comprising administering to a patient a composition comprising at least one of resistin-like molecule a (RELMα), resistin-like molecule β (RELMβ), a regulator of RELMα expression, or a regulator RELMβ expression in a pharmaceutically acceptable formulation and amount sufficient to down-regulate expression of at least one of RELMα or RELMβ to result in at least one of reduced acid secretion, reduced leukocyte accumulation, reduced mucus production, reduced cell proliferation, reduced collagen deposition, or reduced fibroblast accumulation for enhanced repair of the inflamed tissue.

27. The method of claim 27 wherein the regulator of RELMα or RELMβ expression is a Th2 cytokine.

28. The method of claim 27 wherein the regulator of RELMα or RELMβ expression is at least one of IL-4 or IL-13.

29. The method of claim 27 wherein the regulator of RELMα or RELMβ expression further comprises signal-transducer-and-activator-of-transcription (STAT6.)

30. The method of claim 30 wherein the regulator is at least one of a small molecule activator of STAT6, a STAT6 oligonucleotide, or an activator of STAT6 transcription.

31. The method of claim 27 wherein the inflamed tissue is at least one of airway, lung, trachea, bronchoalveolar lavage fluid, skin, eyes, throat, or nose.

32. The method of claim 27 wherein the patient is allergic or asthmatic.
